(19) United States
(12) Patent Application Publication
(10) Pub. No.: US 2026/0232862 A1
(43) Pub. Date: Aug. 13, 2026

(54) METHOD FOR PRODUCING NEGATIVE ION DEVICE FOR PREVENTING AND TREATING AIRBORNE TRANSMISSION VIRUS INFECTIONS

(71) Applicant: Jia XU, Ningbo (CN)

(72) Inventor: Jia XU, Ningbo (CN)

(21) Appl. No.: 19/155,196

(22) PCT Filed: Feb. 27, 2024
(86) PCT No.: PCT/CN2024/078766

§ 371 Date: Aug. 8, 2025

(30) Foreign Application Priority Data
Mar. 11, 2023 (CN) 202310230908.0

(51) Int. Cl. A61L 9/22; F24F 8/30; F24F 8/60

(52) U.S. Cl. CPC A61L 9/22; F24F 8/30; F24F 8/60; A61L 2209/132; A61L 2209/134

(57) ABSTRACT

The present invention discloses a method for producing negative ion device for preventing and treating airborne transmission virus infections. Specifically, water is added to tourmaline crushed stones to moisten them but not completely submerge them, and additional wind pressure is applied to the surface wetted tourmaline crushed stones. Tourmaline crushed stones generate piezoelectric electrodes, causing the surrounding water molecules to ionize and generate a large amount of hydroxyl and hydrogen ions. Hydrogen ions, due to their weaker electron acquisition ability compared to oxygen molecules and being the lightest substance, quickly disperse after being sent out by the fan, and the content in the environment is extremely low. After hydroxyl ions give electrons, water and oxygen are generated, and the electrons they provide are combined with oxygen molecules blown in by the fan and the oxygen molecules generated by their own reaction to form true high concentration negative oxygen ions.

### TECHNICAL FIELD

The present invention relates to the fields of negative ions, air purification and disinfection, medical devices, dispersed oxygen production and supplementation, and mist free humidification technology, in particular to a production method of negative oxygen ion humidifier for preventing and treating airborne transmission virus infections, while also having dispersed oxygen production and supplementation, mist free humidification, air purification and disinfection, other health and therapeutic effects such as relieving respiratory allergies, improving respiratory and circulatory functions, assisting sleep, anti fatigue, antioxidant, clearing free radicals in the body, lowering blood sugar and lipid levels stimulate bone marrow hematopoietic function, increase body repair ability, enhance immunity, etc.

### Background Art

It is generally believed that respiratory viruses, including COVID-19, are mainly transmitted through droplets generated by the infected person when coughing and sneezing or through contact with pollutants. However, an international interdis-ciplinary research team found that aerosols is the most likely main transmission route of most respiratory diseases through its research on super transmission events during the COVID-19 pandemic and many other types of respiratory diseases. This includes SARS virus, Middle East respiratory syndrome coronavirus, influenza virus, measles virus, respiratory syncytic virus, human metap-neumovirus, adenovirus, enterovirus, and rhinovirus that causes the common cold. The omicron mutant of COVID-19 carries the most positive charge so far, while the ACE2 receptor of COVID-19 carries a lot of negative charges. It is this relatively remote Coulomb force that makes COVID-19 highly infectious; The hemagglutinin protein (HA) responsible for invading of influenza virus is slightly positively charged, which happens to bind to the heavily negatively charged sialic acid receptor through electrostatic attraction. Therefore, the ideal and safe method for preventing and treating respiratory viruses is small particle size and high concentration of negative ions. Small particle size and high concentration negative ions high activity, long migration distance, and easy penetration into the human blood circulation. Can combine with substances such as PM2.5, aerosols, bacteria, viruses, pollen, etc. in the air to cause coagulation and sedimentation. At the same time, it can inhibit bacteria, change or reverse the polarity of the potential inside and outside the virus capsid, and cause the spike of virus to collapse, quickly inactivating the virus, cutting off the electrostatic attraction between the virus receptor binding area and the cell membrane receptor, and preventing the virus from invading human cells. Can accelerate the decomposition of VOCs such as formaldehyde and toluene in the air. It has other health and therapeutic effects such as relieving respiratory allergies, improving respiratory and circulatory function, assisting sleep, anti fatigue, antioxidant, clearing free radicals in the body, lowering blood sugar and lipids, stimulating bone marrow hematopoietic function, increasing body repair ability and enhancing immunity, etc.

### SUMMARY OF INVENTION

### Technical Problem

The existing methods for generating negative ions are mostly obtained by ionizing air with high-pressure negative electrodes, which have problems such as large particle size, short migration distance, inability to perform Brownian motion, low activity, ozone pollution, generating a large amount of static electricity and other issues; There are also methods for producing negative ions using high-energy rare earth or high-energy rare earth composite tourmaline powder, but there are potential issues of nuclear radiation pollution, the problem of persistent release, low concentration and quantity of spontaneous generation of negative ions in tourmaline; There are also methods for preparing negative ions by ionizing water mist, but the actual number of negative ions produced is limited due to the electrons generated by the negative electrode not being timely combined and taken away by sufficient oxygen molecules; At present, diffusion oxygen production and supplementation equipment generally uses the separation of oxygen from the air or electrolysis of water to achieve, which has the problem of high energy consumption and difficult concentration control, resulting in waste; Mist free humidification is commonly prone to mold and bacterial growth, requiring frequent replacement of filters.

### Technical Solution

The purpose of the present invention is to provide a method for producing a negative ion purification and disinfection, dispersion oxygen production and supplementation, Mist free humidification device with the functions of preventing and treating airborne transmission virus infections, as well as other health and therapeutic purposes, to solve the aforementioned technical issues. In order to solve the above-mentioned technical problems, the present invention provides the following technical solutions (FIG. 1): by installing an automatic differential pressure water replenisher, the tourmaline crushed stones that are tightly laid or bonded to the base are added with water for replenishment, positioning the highest replenishment water level close to submerging the upper layer of tourmaline crushed stones, and utilizing the capillary phenomenon generated by the surface tension of water to keep the upper layer of tourmaline crushed stones moist but not completely submerged by water; Using a weak current fan to continuously supply wind into the box, the moist tourmaline crushed stones on the upper layer inside the box produces a piezoelectric electrode that is much greater than the spontaneous polarization effect due to sustained wind pressure, ionize the surrounding water molecules, generate a large number of hydrogen ions and hydroxyl ions, hydrogen ions have much weaker electron acquisition ability than oxygen molecules and are the lightest substance, after being sent out by the fan, it quickly dissipates and the content in the environment is extremely low, hydroxyl ions provide electrons to generate water and oxygen, the electrons it provides combine with continuously blowing oxygen molecules and oxygen molecules generated by its own reaction to form small particle size, high concentration negative ions, and it is continuously sent out by the fan, simultaneously perform dispersion oxygen production and supplementation, as well as mist free humidification. The small particle size and high concentration negative ions sent out by the above scheme can accelerate the decomposition of harmful VOCs such as formaldehyde and toluene in the air, it can combine with substances such as PM2.5, aerosols, bacteria, viruses, pollen, etc. in the air to cause coagulation and sedimentation, it can inhibit or kill bacteria, change or reverse the polarity of the potential inside and outside the virus capsid, and cause the spike of virus to collapse, quickly inactivating the virus, cutting off the electrostatic attraction between the virus receptor binding area and the cell membrane receptor, preventing the virus from invading human cells, and it has other health and therapeutic effects such as relieving respiratory allergies, improving respiratory and circulatory function, enhancing human immunity, stimulating bone marrow hematopoietic function, assisting sleep, anti fatigue, clearing free radicals in the body, lowering blood sugar and lipids, and enhancing the body's repair ability, etc.

### Advantageous Effects

Compared to existing technology, the beneficial effect of the present invention is to moisten the upper layer of tourmaline crushed stones by adding water, without completely submerging them, simultaneously apply wind pressure to it, using the piezoelectric polarization effect of tourmaline to ionize surrounding water molecules to produce hydroxyl and hydrogen ions, hydrogen ions quickly disperse from the environment after being blown out, hydroxyl ions provide electrons to generate water and oxygen, the electrons it provides combine with continuously blowing oxygen molecules and oxygen molecules generated by its own reaction to produce a large number of small particle sizes and high concentration negative ions with Brownian motion; It can be used for dispersion oxygen production and supplementation, mist free humidification, air purification and disinfection, quickly sedimentation and in-activate environmental viruses, prevent viruses from invading human cells through electrostatic forces and killing extracellular virus, At the same time, it has other health and therapeutic effects without any toxic side effects, unrestricted user base, especially suitable for individuals with weakened immunity; The production of negative ions and oxygen, mist free humidification are adjusted according to the control of negative ion concentration and humidity in the environment, which will not cause personal injury due to high negative ion concentration or humidity, no issues such as ozone, positive charge accumulation, static electricity, radiation pollution, etc; The tourmaline crushed stones comes with antibacterial, bactericidal, anti mold and purification functions during use, making it easy to rinse and use repeatedly without additional filter consumables, it can be used for an extended lifespan by simply replacing the fan and water bottle accessories; Using water-resistant epoxy resin adhesive to bond a single layer of crushed stones to the base not only saves the amount of crushed stones and facilitates the determination of water level, but also avoids problems such as dirt and bacteria growth in the lower layer of crushed stones.

### DESCRIPTION OF DRAWINGS

FIG. 1 is a three-dimensional view of the present invention.

Component name in the picture:
1. Tourmaline crushed stones tightly laid or bonded to the base; 2. Outer box body; 3. Weak current fan; 4. A pressure differential water replenishment bottle with an external thread on the bottle mouth that can be inverted for retrieval and placement; 5. Pressure differential water replenishment bottle cap with internal thread; 6. Bottle cap water outlet and air inlet; 7. Wind outlet.

### BEST MODE

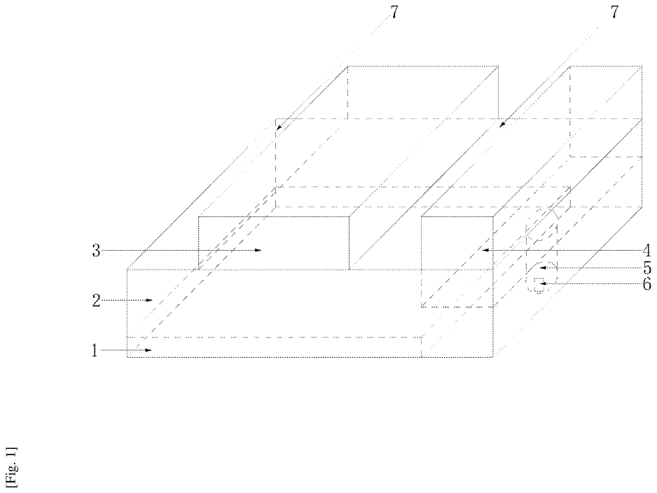

Case 1: as shown in FIG. 1, use water-resistant epoxy resin adhesive to uniformly and tightly bond a certain particle size of chamfered frosted tourmaline crushed stones onto the frosted ABS plastic bottom slightly tilted downwards towards the water replenisher, use an automatic pressure differential water replenisher to add water and replenish water, by utilizing the capillary phenomenon of water, the crushed stones adhered to the bottom are wetted with water but not completely submerged, use a weak current fan to blow wind to the tourmaline crushed stones in the box, produce small particle size, high concentration, highly active negative ions, dispersive oxygen production and supplementation, humidification of air.

### Mode for Invention

Case 2: as shown in FIG. 1, by encapsulating the tourmaline crushed stones tightly laid on the bottom of the plastic box with stainless steel mesh, use a differential pressure water replenisher to add water until the upper layer of tourmaline crushed stones is wetted but not completely submerged, use a weak current fan to blow wind to the tourmaline crushed stone in the box, produce small particle size, high concentration, highly active negative ions, dispersive oxygen production and supplementation, humidification of air.

### INDUSTRIAL APPLICABILITY

When bonding the chamfered and frosted tourmaline crushed stones to the bottom of the frosted ABS plastic box with water-resistant epoxy resin adhesive, a hair dryer can be used to heat the crushed stones and adhesive at the bottom of the box with low air volume from a distance to improve the fluidity of the adhesive. Then, the bottom of the box can be shaken to make the crushed stones evenly and tightly arranged, and fully contact the adhesive. Then, the heating can be continued to accelerate the solidification of the adhesive. The far-infrared emission function of tourmaline crushed stones can be utilized to slowly release waste heat, and intermittent blowing and heating can accelerate solidification. After sufficient curing, flip the box and use a hair dryer to blow out excess crushed stones that have not been glued, which will help further heat and solidify. Then the next assembly operation can be carried out, suitable for industrial large-scale production. Automatic differential pressure water replenisher, manufactured by bottle extrusion blow molding technology, combined with matching threaded bottle caps, can be produced and sold in the form of a complete machine or accessories. It is fixed in the ABS bottom box grid by reverse buckle, convenient and practical. The weak current fan module can be equipped with plastic baffles to reduce the backflow of air and increase air circulation and exchange, greatly improving overall efficiency. It is designed as a flexible disassembly unit for easy cleaning and replacement.

### FREE TEXT OF SEQUENCE LISTING

Input the paragraph which describes the free text of sequence listing here.

1. A method for producing negative ion device for preventing and treating airborne transmission virus infections, its characteristic is to add water to the tourmaline crushed stones to moisten them but not completely submerge them, and apply additional wind pressure to the surface wetted tourmaline crushed stones, utilizing the piezoelectricity of tourmaline to generate piezoelectric electrodes that are far greater than spontaneous polarization effects, ionizing water molecules on the surface of crushed stones generates a large amount of hydroxyl and hydrogen ions, hydrogen ions have much weaker electron acquisition ability than oxygen molecules and are the lightest substance, after being sent out by the fan, it quickly dissipates and the content in the environment is extremely low, hydroxyl ions provide electrons to generate water and oxygen, the electrons it provides combine with continuously blowing oxygen molecules and oxygen molecules generated by its own reaction to form small particle size high concentration negative ions, and it is continuously sent out by the fan, simultaneously perform dispersion oxygen production and supplementation, as well as mist free humidification, the small particle size and high concentration negative ions obtained through green methods are used to accelerate the decomposition of harmful VOCs such as formaldehyde and toluene in the air, combine with substances such as
PM2.5, aerosols, bacteria viruses pollen, etc. in the air to cause coagulation and sedimentation, inhibit or kill bacteria, change or reverse the polarity of the potential inside and outside the virus capsid, and cause the spike of virus to collapse, quickly inactivating the virus, cutting off the electrostatic attraction between the virus receptor binding area and the cell membrane receptor, preventing the virus from invading human cells, and it has other health and therapeutic effects such as relieving respiratory allergies, improving respiratory and circulatory function, enhancing human immunity stimulating bone marrow hematopoietic function, assisting sleep anti fatigue, clearing free radicals in the body, lowering blood sugar and lipids, and enhancing the body's repair ability.

2. (canceled)

3. (canceled)
